SEQUENCE LISTING (17 sequences)

SEQ ID NO: 1 - DNA, 20 bp, Homo sapiens
ggttatcgaccaggtgctcc

SEQ ID NO: 2 - DNA, 20 bp, Homo sapiens
ggttatcaaccaggtgctct

SEQ ID NO: 3 - DNA, 28 bp, Homo sapiens
gcggtacctaggggttgtctcagggtcc

SEQ ID NO: 4 - DNA, 22 bp, Homo sapiens
gggacagcatattctggttacc

SEQ ID NO: 5 - DNA, 19 bp, Homo sapiens
ctgtctcagggtcctagtg

SEQ ID NO: 6 - DNA, 19 bp, Homo sapiens
ttgtctcagggtcctagtg

SEQ ID NO: 7 - DNA, 21 bp, Homo sapiens
tctgaaagtggaccatatcag

SEQ ID NO: 8 - DNA, 22 bp, Homo sapiens
gggacagcatattctggtttcc

SEQ ID NO: 9 - DNA, 22 bp, Homo sapiens
gggacagcatattctggttacc

SEQ ID NO: 10 - DNA, 21 bp, Homo sapiens
ctctgagtcaggagcgtctcc

SEQ ID NO: 11 - DNA, 27 bp, Homo sapiens
tgctccgtgcatctggcatcccctaac

SEQ ID NO: 12 - DNA, 27 bp, Homo sapiens
tgctccgtgcatctggcatctaaccct

SEQ ID NO: 13 - DNA, 27 bp, Homo sapiens
tgctccgtgcatctggcatccctaacc

SEQ ID NO: 14 - DNA, 27 bp, Homo sapiens
tgctccgtgcatctggcatcctaaccc

SEQ ID NO: 15 - DNA, 27 bp, Homo sapiens
tgctccgtgcatctggcatcaacccta

SEQ ID NO: 16 - DNA, 27 bp, Homo sapiens
tgctccgtgcatctggcatcaccctaa

SEQ ID NO: 17 - DNA, 20 bp, Homo sapiens
tgctccgtgcatctggcatc

(19) United States
(12) Patent Application Publication
(10) Pub. No.: US 2004/0265815 A1
(43) Pub. Date: Dec. 30, 2004

(54) Method for determination of telomere length

(76) Inventor: Duncan Martin Baird, Cardiff (GB)

Correspondence Address: KING & SCHICKLI, PLLC, 247 NORTH BROADWAY, LEXINGTON, KY, 40507, US

(21) Appl. No.: 10/482,596

(22) PCT Filed: Jun. 21, 2002
(86) PCT No.: PCT/GB02/02855

(30) Foreign Application Priority Data
Jun. 23, 2001 (GB) 0115451.7
Sep. 21, 2001 (GB) 0122755.2

(51) Int. Cl. C12Q 1/68; C12P 19/34

(52) U.S. Cl. 435/6; 435/91.2

(57) ABSTRACT

A method is described for determining telomere length of mammalian chromosomal DNA, which method comprises the steps: (a) annealing the 3′ end of a single-stranded oligonucleotide (hereinafter referred to as a ‘telorette’) to a single-stranded overhang of the telomere comprising the G-rich telomere strand (comprising TTAGGG repeat sequences) and covalently binding the telorette to the 5′ end of the C-rich telomeric strand (having CCCTAA repeat sequences) thereby forming a ligation product; (b) amplifying the ligation product formed in step (a) to form a primer extension product; and (c) detecting the length of the primer extension product(s) of step (b). Step (b) is preferably carried out by PCR using: (i) a first primer capable of annealing to a telomere-adjacent region of DNA but which first primer is not capable of annealing to the C-rich telomeric repeat sequence (CCCTAA); and (ii) a second primer (hereinafter referred to as a ‘teltail’ primer) identical to the 5′ end sequence of the telorette of step (a) which amplification is effected under conditions such that the first primer hybridises to the C-rich telomeric strand (comprising the CCCTAA repeats) and is extended to form a first primer extension product; and the teltail primer hybridises to the first primer extension product and is extended to form a second primer extension product. Also described are specific primers, including telorettes and teltails, for carrying out the method; a kit for use in the method; and the use of the method in determining telomere length and assessing biological conditions associated therewith.

The present invention relates to a method for the measurement of mammalian telomere length, in particular, for the measurement of human telomere length. The invention further relates to primers and other reagents for use in the method and to a kit for carrying out the method, which comprises one or more of said primer(s) or reagent(s).

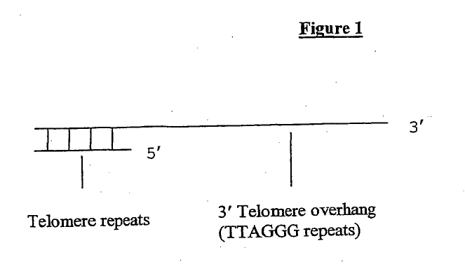

Telomeres are DNA structures that cap the ends of eukaryotic chromosomes, and are important in maintaining chromosome stability and function. In humans, telomeres are composed of many kilobases, eg as many as 20 kb, and consist of the DNA sequence motif TTAGGG, tandemly repeated. These repeats are arranged such that the G-rich strand runs 5′ to 3′ towards the end of the chromosome and sometimes extends beyond the 5′ end, resulting in a single-stranded overhang comprising TTAGGG repeats (illustrated schematically in FIG. 1, hereinbelow).

During cell division, as the chromosomes divide, telomere sequences are lost from the end. The enzyme telomerase can synthesise TTAGGG repeats de novo at the terminus, thus extending the DNA and preventing shortening. This enzyme is largely inactive in somatic cells, where the telomere therefore shortens with each division. The degree of telomeric sequence loss depends upon the age, replicative history and telomerase activity of a particular tissue. Consequently, telomeric loss can be correlated with the introduction of the biochemically active but non-dividing state known as cellular senescence.

In contrast, telomerase is active in germ line cells, resulting in the maintenance of telomere length in the germ line for subsequent generations. In addition, the enzyme is also active in malignant tumour cells and the stem cells of some proliferative somatic tissues.

There are a number of possible mechanisms for the loss of telomere DNA during ageing, including incomplete replication, degradation of termini and unequal recombination coupled to selection of cells with shorter telomeres. Cellular senescence may have evolved as a tumour protection mechanism such that, in order for a tumour to progress to malignancy, the telomerase enzyme has to be activated so that the tumour cells can continue to divide. The corollary of this is that, in normal somatic cells, the loss of telomeric DNA as a consequence of telomerase repression may lead to the age-related accumulation of senescent cells. This, in turn, may underlie some age-related pathologies, such as age-related degeneration of the intravertebral discs (fibrocytic cell senescence), atherosclerosis (vascular endothelial cell senescence), ocular degeneration (retinal pigmented epithelial cell senescence) and immuno-senescence (T-cell senescence), as well as problems with wound healing (fibroblast senescence).

Knowledge of telomere length and the presence or lack of telomerase activity can alsoprovide information about the replicative history and proliferative potential of cells. Thus, there is a need for an accurate method for determining telomere length using only in the order of 1000 cells or even determining the telomere length of a single DNA molecule.

Restriction enzyme digestion of genomic DNA and Southern hybridisation to a TTAGGG repeat containing probe reveals the average size of the terminal restriction fragments (TRFs) from all chromosome ends simultaneously. TRF analysis is currently the method of choice for estimating telomere length from DNA samples in the majority of organisms. However, these tests therefore suffer from various disadvantages, including: that they require a minimum of 200,000 cells to obtain sufficient DNA (1 μg); and they determine only the average length of all telomeres in one go and cannot provide specific information on sequence content and length of single telomeres. In addition, these tests are relatively inaccurate, as it is not the exact TTAGGG content that is measured, since the restriction fragments generated include varying lengths of DNA flanking the telomere. An alternative method, Q-FISH, uses quantitative (TTAGGG)n fluorescence in situ hybridisation to metaphase chromosomes. This approach has the advantage that, unlike TRF analysis, it can determine the TTAGGG repeat content at all the chromosome ends in isolation. However, only relatively small datasets can be produced and it requires good metaphase chromosome preparations, which restricts the analyses to cells that can proliferate well in culture.

U.S. Pat. No. 5,741,677 describes a method for measuring the average length of telomeres in a sample comprising a cell or tissue, which method involves contacting the 3′ end of a telomere with an oligonucleotide linker under conditions such that the linker becomes covalently bound to the 3′ end of the telomere. The DNA is then amplified using, for example, the polymerase chain reaction (PCR) using a first primer complementary to the oligonucleotide linker and a second primer complementary to a sub-telomeric region of a chromosome, ie a portion of the chromosome which is 5′ to the telomere sequence. The amount by which the first and/or second primer has been extended to form extension products is then measured, as a result of which the average telomere length is determined.

U.S. Pat. No. 5,834,193 describes a method for measuring telomere length, which method comprises contacting denatured chromosomal DNA that has not been fractionated by gel electrophoresis with a labelled probe having a sequence complementary to a telomere repeat sequence, and under conditions such that the probe hybridises specifically to telomeric DNA. The amount of bound probe is then measured, and correlated relative to a control of known telomere length.

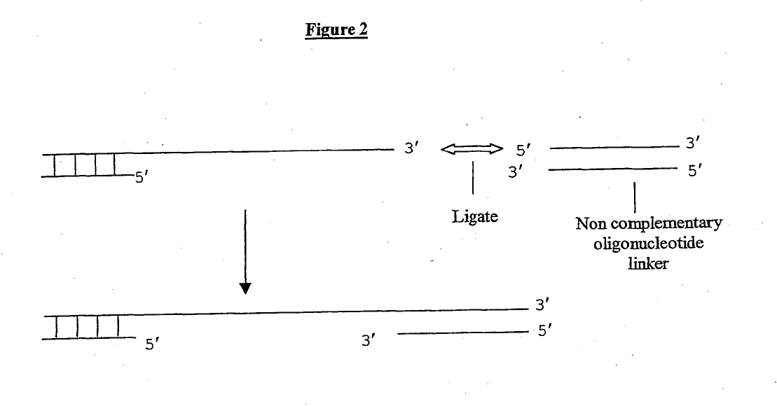

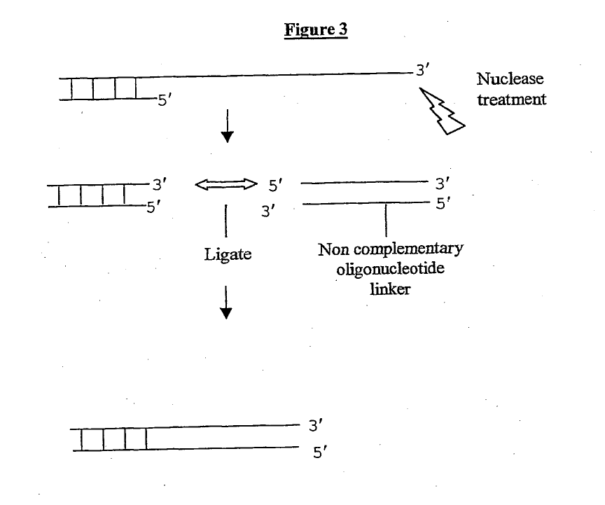

Both patent specifications describe the ligation of a non-complementary oligonucleotide linker to the 3′ end of the telomere (illustrated schematically in FIG. 2, hereinbelow). In U.S. Pat. No. 5,834,193 is stated that “any double-stranded linker can be used, so long as the linker sequence differs from the telomeric repeat sequence”. In a preferred embodiment, the specification discloses initial nuclease treatment to render the terminus blunt-ended, which is followed by the blunt-ended ligation of the oligonucleotide linker sequence, as shown in FIG. 3 hereinbelow.

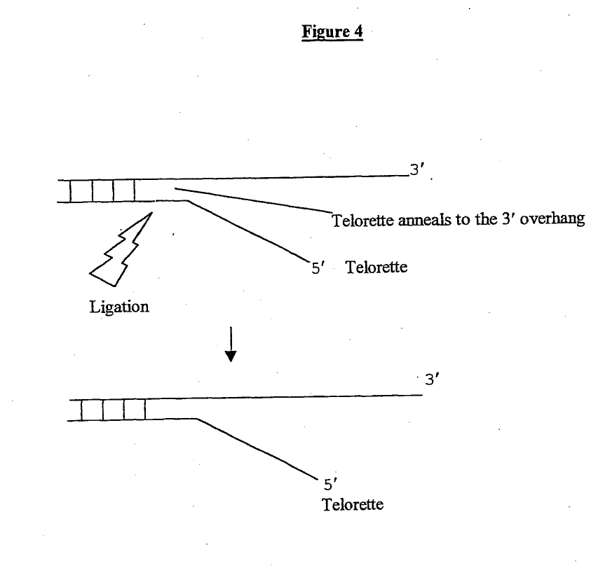

It has now been found that, by ligating to the 5′ end of the C-rich telomeric strand, a single-stranded oligonucleotide linker, which oligonucleotide linker is homologous to the tandemly repeated human telomeric sequence TTAGGG and at the 5′ end a sequence identical to a primer suitable for amplification, eg by PCR, and amplifying the ligated product, it is possible to determine telomere length using smaller numbers of cells (by about two orders of magnitude) than presently available methods. In addition, it may be possible to amplify from single DNA molecules and therefore determine the exact number of TTAGGG repeats in the telomere. (Schematically illustrated in FIG. 4).

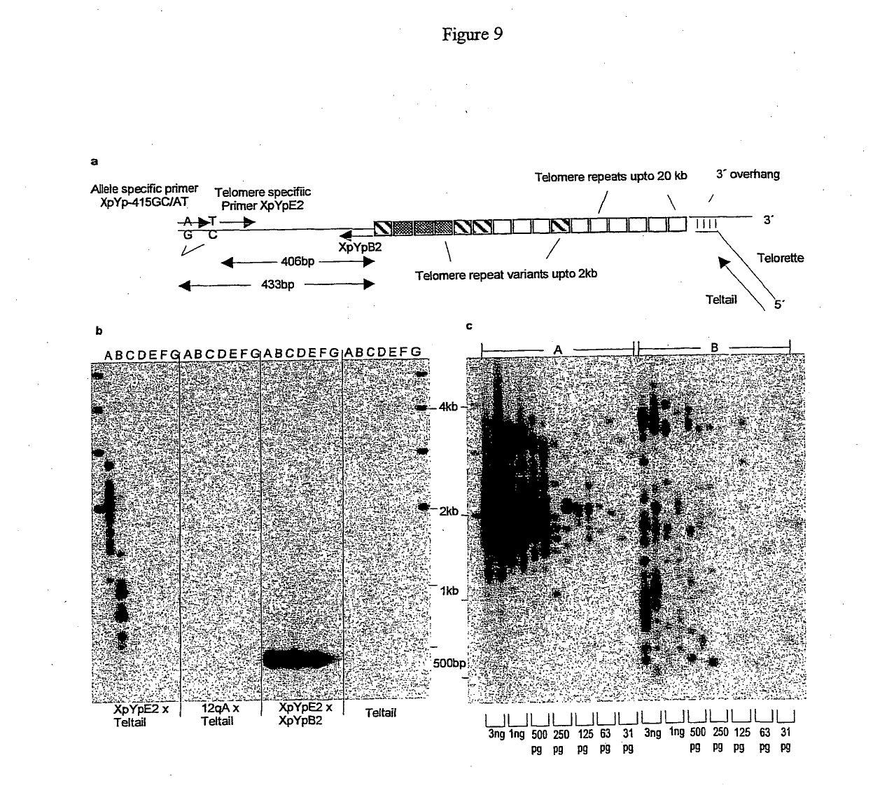

Specifically, it has been found possible to design oligonucleotide linkers (‘telorettes’ 1-6, described below) to anneal to the G-rich telomere strand of the 3′ terminal overhang at any one of the six possible positions within the 6 bp telomere repeat units. These specific ‘telorette’ linkers contain 7 bases of telomere homology and a 5′ non-complementary tail of 20 nucleotides. An additional oligonucleotide primer (‘teltail’) was designed to be identical in sequence to the 5′ tail of the ‘telorette’ linkers. Following ligation of the ‘telorettes’ to the 5′ end of the C-rich telomeric strand, PCR, for example, results in exponential amplification of specific telomeric products, only in the presence of strand extension from a telomere adjacent primer synthesising the complement to the 5′ tail of telorette, and facilitating annealing and second strand synthesis from the teltail primer (FIG. 9).

This technique may advantageously be applied to any chromosomal telomere, provided that the DNA sequence flanking the telomere is known. It is also applicable to other mammalian species, besides humans, provided that the chromosomes have a terminal 3′ overhang; that the telomere does not exceed 25 kilobases; and that the sequence flanking the telomere is known.

Therefore, according to one aspect of the invention, there is provided a method for determining telomere length of mammalian chromosomal DNA, which method comprises the steps:

(a) annealing the 3′ end of a single-stranded oligonucleotide (hereinafter referred to as a ‘telorette’) to a single-stranded overhang of the telomere comprising the G-rich telomere strand (comprising TTAGGG repeat sequences) and covalently binding the telorette to the 5′ end of the C-rich telomeric strand (having CCCTAA repeat sequences) thereby forming a ligation product;

(b) amplifying the ligation product formed in step (a) to form a primer extension product; and

(c) detecting the length of the primer extension product(s) of step (b).

Step (a) is preferably carried out under conditions such that the covalent binding occurs by ligation. Preferably, the conditions allow for annealing and ligation to be carried out in the same step (one pot reaction).

Preferably, step (b) is carried out, for example by a polymerase chain reaction (PCR), using:

(i) a first primer capable of annealing to a telomere-adjacent region of DNA but which first primer is not capable of annealing to the C-rich telomeric repeat sequence (CCCTAA); and

(ii) a second primer (hereinafter referred to as a ‘teltail’ primer) identical to the 5′ end sequence of the telorette of step (a)

which amplification is effected under conditions such that the first primer hybridises to the C-rich telomeric strand (comprising the CCCTAA repeats) and is extended to form a first primer extension product; and the teltail primer hybridises to the first primer extension product and is extended to form a second primer extension product.

The teltail primer may itself comprise the whole telorette sequence, but preferably comprises a shorter sequence, which is unique and identical to the 5′-end of the telorette sequence, but, in either case, is not complementary to the telorette.

To assist in the understanding of the invention, the following terms used herein are defined as follows:

‘Primer’ means an oligonucleotide designed to hybridise (bind) to a target nucleic acid, which hybridised sequence can then be extended by the addition of nucleotide(s) or an oligonucleotide(s). A primer is typically extended by the action of a polymerase or ligase. Typically, an oligonucleotide primer will be 8 or more nucleotides in length, preferably 12 to 15, but may be 20 or more nucleotides in length.

‘Sub-telomeric DNA’ or ‘sub-telomeric region’ is used to mean the same as ‘telomer-adjacent’ DNA or ‘telomere-adjacent’ region. This signifies chromosomal DNA located adjacent to (preferably in the range of from 100 to 500 bp but can be up to 20 kb, although is preferably up to 1 kb or even 2 kb, from) the tandem telomeric repeats of the telomeric DNA. Accordingly, the first primer does not become (after amplification) located in the telomeric DNA itself. Primer extension is effected under conditions such that the first primer anneals to the same strand that comprises the C-rich telomere repeats (CCCTAA). Sub-telomeric DNA generally contains various classes of repetitive elements, such as ‘mini-satellites’, which are often interspersed with telomere and degenerate telomere repeat sequences.

‘Telomeric DNA’ or ‘telomeric region’ means chromosomal DNA located at the ends of the chromosomes and which consists of a tandemly repeated sequence of nucleotides. In humans, the telomeric region comprises 5′-TTAGGG-3′ repeats and the corresponding complementary sequence. The proximal 2 kb of human telomeres comprise, in addition to the canonical telomere repeat sequence TTAGGG, telomere repeat variants, such as TGAGGG, TCAGGG and TTGGGG. The telomeric regions of other species differ with respect to the telomeric repeat sequence and overall telomere length. Nevertheless, the telomere sequences are conserved amongst mammals. Therefore the same telorette and teltail primers could be used in the method of the invention, whether applied to humans or other mammals. Only the telomere-adjacent sequences would be required to specifically analysed at each chromosome end of the species of interest. As with humans, there are very few of these sequences that have been characterised in other mammalian organisms. The XpYp telomere-adjacent sequences in chimps, gorillas and orangutans have been analysed, of which only orangutans show evidence that the XpYp sequence is immediately adjacent to a telomere. Therefore, a specific primer, XpYpEorang (sequence hereinbelow), may be capable of determining XpYp telomere length in the orangutan.

‘Proximal’ and ‘distal’ take their usual meanings in the art, indicating ‘near the centre’ or ‘at the end’, respectively, of the chromosome. Hence, the proximal 2 kb of human telomeres signifies the 2 kb of human telomere DNA furthest towards the centromere (the centre of the chromosome). Conversely, the distal 2 kb refers to the DNA nearest the end of the chromosome.

‘Telorette’ means a single-stranded oligonucleotide comprising, at the 3′ end, a sequence of nucleotides that is homologous to the telomeric repeated sequence (in humans, TTAGGG). The 3′ sequence should have a region of homology sufficiently long to allow specific hybridization to the telomere sequence, in particular, to allow annealing under the conditions used, for example the relatively high ligation temperatures employed when using PCR (at least about 35° C.), and sufficiently short to prevent the telorette from hybridizing to the internal repeats during the subsequent amplification reaction, for example to prevent annealing during subsequent PCR. Generally, the telorette comprises, at the 3′end, from 6 to 12, preferably from 7 to 10, most preferably from 8 to 9, nucleotide bases which are complementary to and capable of annealing to the G-rich telomeric overhang (comprising the repeated sequence TTAGGG).

The telorette further comprises, at the 5′ end, a sequence of from 15 to 30 bases, preferably 18 to 22 bases, most preferably 20 bases, which are selected so as not to have any known substantial homology to human DNA sequences and to be efficient for PCR amplification. If there were substantial homology to human DNA, then non-telomeric products would arise from the assay, which would defeat an objective of the assay, namely to result in no exponential PCR amplification in the absence of ligation. It is possible to determine the suitability of a potential telorette sequence from this standpoint by interrogating the genome sequence databases available.

The single-stranded oligonucleotide (‘telorette’) used in the method of the invention is therefore distinct from the double-stranded linker described above with respect to the two U.S. patent specifications. First, the telorette comprises, preferably in the first 7 bases, a sequence complementary to the telomeric sequence (TTAGGG in humans). This allows the oligonucleotide to anneal to the telomeric 3′ overhang and provide specificity to the telomeric terminus. The prior art oligonucleotide is not complementary and therefore only ligates to the 3′ end of the telomere without becoming annealed. In addition, the 3′ bases complementary to the telomere are preferably designed to be short enough such that, at the annealing temperatures employed in the subsequent amplification (eg PCR) reaction, the telorette linker would not be capable of initiating strand synthesis. A key difference is therefore that the single-stranded oligonucleotide linker or telorette is designed to target a specific genomic structure, namely the 3′ telomeric terminus. The double-stranded linker described in U.S. Pat. No. 5,834,193 has no specificity for such structures, and therefore it would be capable of being ligated to any DNA break. This is predicted to lead to the production of DNA fragments that have this linker ligated at each end. Such molecules would be amplified with similar—if not greater—PCR efficiencies (particularly if they were shorter than the true telomeric molecules) and would therefore contribute to a significant amount of artefact in the assay. In addition, DNA breaks in the telomere itself would have this linker ligated onto it, and therefore the shorter molecules would be preferentially amplified and there would be no way of distinguishing these molecules from the true telomeric molecules.

Secondly, the remaining 5′ bases of the telorette linker comprise a sequence designed so as not to have any known substantial homology to human DNA sequences and to be efficient for PCR amplification only in the presence of first strand synthesis, initiated from either a primer designed to anneal to the sub-telomeric DNA or the variant repeats in the proximal regions of the telomere. Use of a non-complementary primer (instead of one complementary to the oligonucleotide linker proposed in the U.S. patent) creates the complement to the linker (by first strand synthesis initiated from the telomere-adjacent primer), such that the ‘non-complementary’ primer can anneal and prime second strand synthesis. This increases the specificity of the process, such that only molecules comprising both the telomere-adjacent priming site and the ligated telorette would be capable of exponential amplification. This is in contrast to the U.S. patent method, in which any DNA molecule with the linker ligated at each end would be exponentially amplified.

The mammalian chromosomal DNA can be extracted from cells and tissue using standard laboratory procedure as described by Sambrook J, Fritsch E F and Maniatis T in Molecular cloning: A laboratory manual (second edition) page 9.16. Alternatively, commercially available DNA extraction kits, such as Amersham Pharmacia's Biotech-Nucleon DNA extraction kit, Promega's Wizard® Genomic DNA purification kit, Qiagen-DNeasy® and Cambio-MasterPure™ may be used.

Preferably, the method is used for determining telomere length of human chromosomal DNA.

In step (a) of the method according to the invention, following the extraction of the selected DNA, the telorette is ligated to the 5′ end of the C-rich telomere strand using an appropriate ligase, for example T4 DNA ligase, DNA ligase (E. coli) or any DNA ligase capable of joining, under the reaction conditions, juxtaposed DNA molecules between the 5′-phosphate and 3′-hydroxy groups. Since the first bases, generally up to 12 in total, of the 3′ end of the telorette are homologous to the human telomeric sequence TTAGGG, they will also anneal, as required, to the G-rich telomeric strand.

The exact sequence of the telorette linker can vary only in the position within a TTAGGG repeat that the primer is designed to anneal to. For example, in the method of Example 1 hereinbelow, two different telorettes have been used, of which Telorette 2 is preferred and the results shown therefore relate to Telorette 2 and not Telorette 1. Other variations of this linker could be envisaged, as shown in Examples 3 and 4 hereinbelow. The efficiency of a telorette is defined as the percentage of amplifiable molecules per haploid genome, calculated by Poisson analysis of single molecule dilutions as described hereinbelow. In order to improve the efficiency, each telorette could be ligated separately and the ligations combined prior to amplification (eg by PCR). It may be advantageous to use a set of six telorettes, which are designed to hybridize to any part of the telomere repeat sequence TTAGGG. The sequence at the 3′ end of the six members of the set of telorettes would therefore include the following sequences: AATCCC, ATCCCA, TCCCAA, CCCAAT, CCAATC and CAATCC. Corresponding sequences are used for species other than humans.

This ligation reaction using only the telorette is preferably carried out at a relatively high ligation temperature of from 35° C. to 37° C. in order to allow the specific ligation of the telorette to the 5′ end of the C-rich telomeric strand and without the formation of other non-specific ligation products that may be produced at lower temperatures. Following ligation, the ligase enzyme is heat-inactivated by raising the temperature to, for example, 70° C., for a period of time, such as 15 minutes.

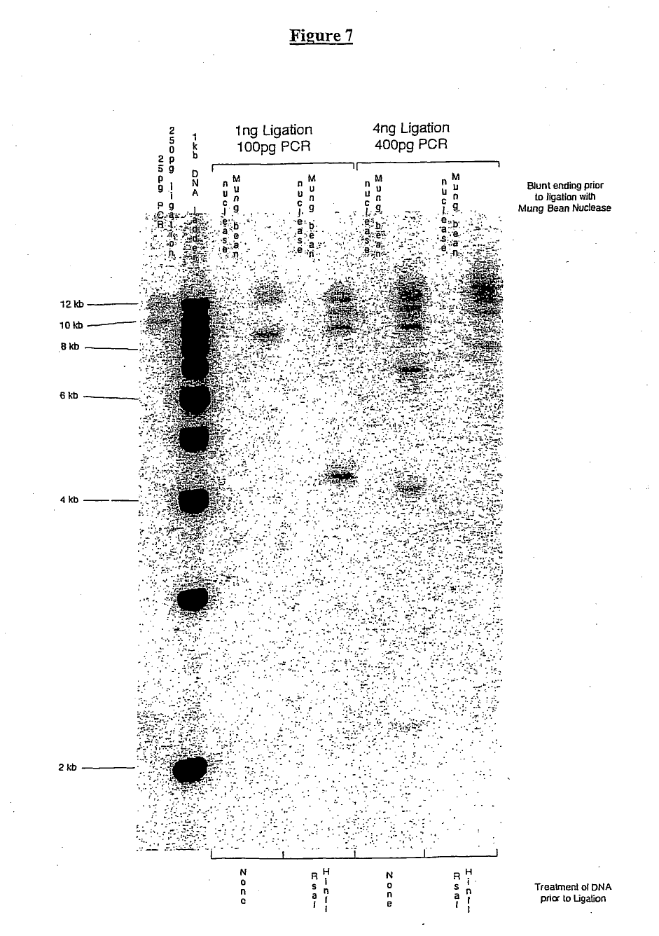

As a negative control for the ligation and the fidelity of the subsequent (PCR) amplification, the 3′-overhang can be rendered blunt-ended by an appropriate nuclease, for example Mung bean nuclease (as illustrated in FIG. 7 and FIG. 9 hereinbelow). This prevents ligation of the telorette linker to the 5′ end of the telomere and demonstrates the requirement for base-pairing of the telorette primer to the 3′ strand of the telomeric overhang in order for the ligation reaction to take place.

In step (b) of the method according to the invention, following ligation, the resultant ligated product is amplified, such as by PCR, preferably using long-range PCR conditions, to ensure amplification of long telomeres (see Cheng, “Efficient PCR of Long Targets”, New Horizons in Gene Amplification Technology: New Techniques and Applications, San Francisco, Calif. (1994)).

To carry out the PCR amplification, a first oligonucleotide primer, which is designed to anneal to either the DNA flanking the specific telomere, for example the 12q or Xp/Yp telomeres, or designed to detect the telomere variant repeats observed within the proximal 2 kb of a human telomere, together with a second primer, the ‘teltail’ primer, the sequence of which is identical to the sequence of the 5′ end of the telorette, as defined above, are used.

It is also possible to use allele-specific PCR primers in the telomere-adjacent DNA to amplify single telomeric alleles in individuals heterozygous for sequence polymorphism in the telomere-adjacent DNA. This can be carried out using primers XpYp-413AT and XpYp-423GC (sequences given hereinbelow), for example at an annealing temperature of about 65-68, preferably about 66.5° C.

Using these primers, exponential PCR amplification will only occur if specific primer extension products are first created from the first primer, namely the primer that anneals to the DNA flanking the telomere or to the telomere variant repeats. Primer extension from this first primer across the ligated telorette sequence will result in single-stranded DNA containing the sequence complementary to the sequence of the ‘teltail’ primer at its 3′ end. Thereafter, in subsequent PCR cycles, annealing of the second ‘teltail’ primer to its complementary sequence followed by primer extension results in exponential PCR amplification of specific telomeric products. The second or ‘teltail’ primer is not capable of annealing and initiating extension products in the absence of complementary strand synthesis initiated from the first primer.

The PCR amplification is preferably carried out using long-range PCR conditions, as described by Barnes W M in Proc. Natl. Acad. Sci. USA 91 2216-2220 (1994), which was the first description of the use of specific conditions to limit DNA damage during the cycling process by maintaining the pH via the use of buffering agents and limiting the denaturation temperature by the use of co-solvents, such as glycerol, thereby allowing denaturation at lower temperatures and for shorter times. In addition, the use of a mixture of standard thermostable polymerases and those with proof-reading ability allowed long PCR products to be generated. Long-range PCR allows the amplification of long telomeres, such as those found in human sperm, and ensures that all telomeres are amplified, thereby enabling the full spectrum of telomere lengths to be observed.

In addition, both “hotstart” and “touchdown” cycling procedures may be used in order to maximise the specificity of the reaction. “Hotstart” PCR, as described by Chou Q et al in NAR 20 1717-1723 (1992), suppresses mis-priming artefacts, can increase yield and the consistency of the reactions. The reactions components are physically separated or chemically inactivated prior to the initial denaturation step. The technique probably works by preventing strand extension of inappropriately annealed PCR primers prior to the initial denaturation. “Touchdown” PCR protocols, as described by Don R H et al in NAR 19 4008 (1991), can increase the specificity and consequently the yield of the reaction. It employs a cycling regime whereby the annealing temperature is set high and reduced in subsequent cycles to a final annealing temperature at which several cycles are performed. This protocol is thought to favour the production of the specific product over spurious products formed by mispriming in the initial cycles of the PCR reaction. The “hotstart” procedure can use commercially-available heat-activated DNA polymerases or wax beads to facilitate the separation of the reaction components until the reaction is heated. Alternatively, the appropriate reaction components can be added after the initial PCR denaturation step.

Preferably, hotstart and touchdown techniques are not used, in favour of PCR amplification occurring with annealing at an annealing temperature of about 65° C. Although PCR amplification is preferred, other amplification methods may also be used.

Following PCR, the DNA primer extension products may be resolved using agarose gel electrophoresis followed by transfer from the gel onto a nylon membrane using standard laboratory Southern blotting procedures or in-gel hybridisation techniques. The DNA fragment may then be detected by hybridisation with a DNA probe containing the sequence of the sub-telomeric DNA of the telomere of interest. Alternatively, the DNA fragment may be detected by hybridisation with a TTAGGG repeat probe which may be labelled with, for example, ³²P.

Following hybridisation, the hybridised fragment may be detected using standard auto-radiography, phospho-imaging or fluor-imaging. The size of the fragments is calculated by reference to DNA size standards, such as a 1 kb ladder (size range 1-12 kb) and a 2.5 kb ladder (size range 2.5-35 kb+).

In order to determine the size of hybridised fragments, an additional hybridisation probe may be included in order to detect the DNA size ladders so that they can be observed along with the PCR products by phospho-imaging. Detection by phospho-imaging has the advantage of allowing computer-based calculation of the fragment sizes.

The method for calculating the size of the fragments is a standard laboratory technique whereby the distance that the DNA size markers have migrated through the gel is plotted against the molecular weight of the markers. The resulting curve is then used to determine the size of the fragment of interest.

When amplifying single DNA molecules, calculating the size of the telomere length using this method is straightforward, as a single band will be observed. However, if more than a single DNA molecule is amplified, a smear of hybridising fragments is obtained. This is because telomere lengths are not homogenous and tend to vary around a mean telomere length; this is presumably due to the random nature of the loss of telomere repeat sequences. In this case, the mean telomere length of the smear is calculated. Using suitable computer software, for example Molecular Dynamics ImageQuant software, volume analysis may be carried out using a grid of, for example, 1×30 rows placed over each lane of the gel.

These data may then be imported into a spreadsheet to create a numerical representation of the autoradiograph. A grid is also placed over the DNA markers, and the grid position of each molecular weight marker is determined and the data entered into the spreadsheet to determine a molecular size curve. The corresponding size of each position on the grid is calculated from the molecular size curve. The Mean Telomere Length (MTL) is calculated using the formula MTL=Σ(MWᵢ×ODᵢ)/Σ(ODᵢ) where MW=the molecular weight at grid position i and ODᵢ is volume of position i. This is a standard method of calculating MTL and is described by Kruk et al in PNAS 92 258-262 (1995); however, alternative formulae may be used, such as MTL=Σ(ODᵢ)/Σ(ODᵢ/MWᵢ) as described by Harley et al in Nature 345 458-60 (1990) when a TTAGGG repeat-containing probe is used in the Southern hybridisation step.

The final step is to subtract from the MTL or the single band length, the distance from the point of annealing of the oglionucleotide primer in the telomere adjacent DNA to the start of the telomere. This allows the amount of the telomere repeats to be determined without telomere adjacent DNA. This is not possible with standard telomere length analysis where there is an unknown and potentially variable amount of telomere adjacent DNA present on each terminal restriction fragment.

The assay of the present invention is so sensitive that single DNA molecules can be amplified and their telomere length determined. Indeed, the sensitivity of the assay is such that a smear correlating to telomere length is generally obtained when more than a single DNA molecule is amplified. Dilution of samples for analysis may improve performance of the assay. Serial dilution of samples may be carried out to the point that single molecules are amplified The DNA containing samples may be diluted, preferably serially diluted, either before the ligation reaction or the ligation reaction mixture itself may be diluted. However, if the technique is to be used to quantify in detail the extent of heterogeneity in telomere length (ie the additional fragments outside of the average telomere length), a more sophisticated series of experiments is required. This is because serial dilutions from a known amount of DNA to the single molecule level can be inaccurate, and the ligation reaction is not 100% efficient and will vary between samples. Therefore, in order for single molecule telomere length analysis to allow the quantification of samples and comparisons with different samples, the number of amplifiable molecules in the diluted DNA should be determined. This may be carried out by the dilution of the DNA to the point at which not all the PCR reactions contain any amplifable molecules. A large number of PCR reactions are performed (60 or more), and the number of positive and negative reactions is quantified. Poisson analysis is then used to allow the number of amplifable molecules per quantity of DNA in the original sample to be determined. These experiments are similar to those described by Jeffreys A J et al in Nature Genetics 6 136-145 (1994) for the quantification of single molecule efficiency for PCR in the DNA flanking some ‘mini-satellite’ loci.

With conventional telomere analysis techniques, only the average telomere length can be determined from a population of at least 10⁵, and it is not possible to determine the telomere length of single molecules. Using the method of the present invention, single telomeres can be determined and thus the full extent of telomere length heterogeneity may also be determined. For example, using the method of the present invention in investigations of standard telomere length, analysis of the human germline reveals that telomere length is 12 kb and telomere lengths up to 20 kb have been observed in genomic DNA. However, single molecule analysis shows many telomeres much shorter than 12 kb. For example, in work described in more detail in Example 2 below, the majority of bands were found to be about 12 kb but additional bands of about 2.2 kb, 4 kb and 7 kb were observed. Telomeres of that length were not observed using conventional telomere length analysis.

Amplification products are not detected below the size of the minimum possible telomere length 406 bp (FIG. 9). This, coupled with the quantal nature of the amplification (ie that the single bands have a similar intensity), suggests that the amplification reaction is highly telomere-specific, without the apparent generation of PCR artefacts. This view is also supported by the biological data, which is consistent with current understanding of telomere biology. The molecular weight of each band can be determined from the gel, and, as the position of the PCR primer in the telomere-adjacent DNA is known, the exact quantity of telomere repeats can be determined for each telomeric band. Furthermore, when coupled with telomere-variant repeat mapping (TVR-PCR) (as described by Baird, et al in EMBO J 14 5433-5443 (1995); Coleman, et al in Hum. Mol. Genet. 8 1637-1646 (1999); and. Baird, et al in Am. J. Hum. Genet. 66 235-250 (2000)), it is possible to determine the exact sequence composition of each telomeric molecule.

A further advantage of the sensitivity of the present assay is that it allows telomere length determination in cases where only a very small amount of material is available for analysis.

As the method of this invention can involve the use of several pre-existing and commercially available products, any kit for putting it into effect for telomere length determination would not necessarily have to provide all of the reaction components. However, the essential components of an assay kit according to this invention comprise one or more oligonucleotides selected from the ‘telorette’ and ‘tailtail’ sequences, as defined herein; preferably also one or more telomere-adjacent (sub-telomeric) chromosome-specific primers (eg for XpYp only and/or 12q) and/or hybridisation probes.

A full kit could optionally also comprise one or more of the various reaction components described above (although may exclude the reagents necessary for the genomic DNA isolation), such as the ligase, the long-range PCR components and the various buffers for these enzymes. In addition, the kit could optionally include computer software and/or a spreadsheet to allow the calculation of MTL.

Besides its use in researching into telomere length, the method and kit of this invention have applications where measurement of the effect of telomerase inhibitors might be important, such as in assessing potential anti-cancer treatments, in other cancer-related procedures, such as in the analysis of biopsy samples or assessing the effect of stem cells in bone marrow transplantation. The method and kit are suitable in the case of short telomeres, since the method does not bias against these, which can be detected.

The invention further provides specific primers for use in the method of the invention and for incorporation into the kit of the invention, including:

| | |
|---|---|
| XpYp-415GC: | 5′- GGTTATCGACCAGGTGCTCC -3′, |
| XpYp-415AT: | 5′- GGTTATCAACCAGGTGCTCT -3′ |
| XpYpE: | 5′-GCGGTACCTAGGGGTTGTCTCAGGGTCC-3′ |
| 12qA: | 5′-GGGACAGCATATTCTGGTTACC-3′ |
| XpYpEorang: | 5′-CTGTCTCAGGGTCCTAGTG-3′ |
| XpYpE2: | 5′- TTGTCTCAGGGTCCTAGTG -3′ |
| XpYpB2: | 5′- TCTGAAAGTGGACC(AT)ATCAG -3′ |
| 12qB: | 5′-ATTTTCATTGCTGTCTTAGCACTGCAC -3′ |

(XpYpB2 and 12qB point away from the telomere and
can be used in conjunction with XpYpE/E2 and 12qA,
respectively, to generate the telomere adjacent
probes for these telomeres).

| | |
|---|---|
| 7qA | 5′- GGGACAGCATATTCTGGTTTCC -3′ |
| Nitu14eD | 5′- CTCTGAGTCAGGAGCGTCTCC -3′ |
| Telorette1: | 5′- TGCTCCGTGCATCTGGCATCCCCTAAC -3′ |
| Telorette2: | 5′- TGCTCCGTGCATCTGGCATCTAACCCT -3′ |
| Telorette3: | 5′- TGCTCCGTGCATCTGGCATCCCTAACC -3′ |
| Telorette4: | 5′- TGCTCCGTGCATCTGGCATCCTAACCC -3′ |
| Telorette5: | 5′- TGCTCCGTGCATCTGGCATCAACCCTA -3′ |
| Telorette6: | 5′- TGCTCCGTGCATCTGGCATCACCCTAA -3′ |
| Teltail: | 5′- TGCTCCGTGCATCTGGCATC -3′ |

Chromosome specific primers are those that are designed to anneal and prime synthesis in the telomere-adjacent DNA from a specific chromosome end. The hybridisation probe would either be specific to the telomere-adjacent DNA of the chromosome of interest or specific to the telomere repeat sequence TTAGGG. They can therefore also be referred to as telomere specific primers, for example:

| | |
|---|---|
| 12qA | 5′- GGGACAGCATATTCTGGTTACC -3′ |

which would specifically detect the telomere of 12q;

| | |
|---|---|
| 7qA | 5′- GGGACAGCATATTCTGGTTTCC -3′ |

which would specifically detect the telomere of 7q,

| | |
|---|---|
| Nitu14eD | 5′- CTCTGAGTCAGGAGCGTCTCC -3′ |

which would detect a telomere on some copies of 16p and 16q.

It will be evident from the foregoing that the invention further provides the use of a primer or kit as described above in a method of the invention. Further more, there is provided:

(a). A method, kit or primer as described herein, for use in assessing a potential anti-cancer treatment and/or in another cancer-related procedure.

(b). A method, kit or primer as described herein, for use in the analysis of a biopsy sample or assessing the effect of stem cells in bone marrow transplantation.

(c). A method, kit or primer as described herein, for use in assessing telomere dynamics with age.

(d). A method, kit or primer as described herein, for use in assessing the effect of modulating telomerase activity.

(e). A method, kit or primer as described herein, for use in assessing, treating or diagnosing male infertility.

(f). A method, kit, primer or use as described herein, substantially as hereinbefore described.

The invention will now be further illustrated with reference to the following Examples.

### EXAMPLE 1

Material and Methods

All the materials used for DNA extraction were molecular biology grade reagents (i.e. tested for the absence of DNase and RNase) and were purchased from Sigma-Aldrich Company Ltd. Poole, Dorset UK.

DNA Extraction and Quantification

1. Cell lysis in a 500 μl solution of 100 mM NaCl, 10 mM Tris-HCl (pH 8.0) and 0.5% SDS

2. Digestion in the above solution with Proteinase K at a final concentration of 1 μg/ml at 50° C. overnight.

3. Extraction with phenol/chloroform/isoamyl alcohol (25:24:1) (500 μl) twice, spin 13,000 rpm and remove aqueous phases.

4. Ethanol precipitation, in presence of 300 mM sodium acetate and 3 volumes of 100% ethanol (Absolute Alcohol A.R. Quality, obtained from Hayman Limited, Eastways Park Witham, Essex CM6 3YE).

5. Spin 13,000 rpm 5 minutes and remove supernatant.

6. Wash DNA pellet with 70% ethanol and air dry.

7. Re-suspend DNA pellet in 50 μl of 10 mM Tris-HCI (pH 8.0).

8. Quantify the DNA concentration by fluorometry or other methods.

Ligation

The ligation reaction was carried out as follows, using T4 DNA ligase and reaction buffer as supplied by Amersham/Pharmacia.

1. A 5 μl reaction containing 10 ng or less of genomic DNA, 1× manufacturer's ligase reaction buffer and 0.9 μl of a 10 μM solution of the oligonucleotide linker “Telorette”.

2. The reaction is heated to 60° C. for 5 mins, and cooled to 35° C., this is carried on a standard laboratory thermal cycler.

3. Once the reaction was at 35° C., 5 μl of the following solution was added to each reaction (1× manufacturer's ligase reaction buffer containing 0.5 unit of T4 DNA ligase)

4. The reaction was incubated at 35° C. for between 6 to 12 hours, and the enzyme heat-inactivated at 70° C. for 15 mins.

PCR Amplification

The following PCR reaction was carried out using commercially-available long-range PCR reaction components (in this case, the Extensor Hi-Fidelity PCR kit supplied by ABGene) and a ‘hotstart’ procedure in which the reaction components were added after the initial denaturation. The long-range PCR reaction can use any commercially-available system procedure, using additives such as glycerol to allow a lower denaturation temperature, the addition of Tris base to maintain a high pH and a mixture of Taq polymerase and a polymerase with proof-reading ability (eg Pwo, Pfu and Vent).

For a 20 μl PCR reaction

1. Set up an initial reaction containing 1× long range reaction buffer (in this case the Extensor Hi-Fidelity PCR kit buffer (this buffer contains MgCl₂ at a concentration of 22.5 mM). The final MgCl₂ concentration is adjusted to 6 mM by the addition of 0.6 μl of a 25 mM stock of MgCl₂, Oligonucleotide primers “Teltail” and the appropriate subtelomeric primer, in this case “XpYpE” (for analysis of the XpYp telomere) or “12 qA” (for the analysis of the 12q telomere) are added to the mixture to a final concentration of 2 μM. Finally, 1 μl of the ligation reaction is added to the reaction mixture.

2. The reaction mixture is heat denatured at 94° C. for 1 minute and cooled to 80° C., and 10 μl of a mixture pre-warmed to 80° C. containing 1× reaction buffer 1, NTPs at a concentration of 0.6 mM and 1 unit of the ABGene Taq/Pwo mix.

3. Therefore, the final concentration of the reaction components are as follows: MgCl₂, 3 mM; oligonucleotide primers, 1 μM and NTPs 0.3 mM.

4. The reaction is cycled as follows: 68° C. 10 minutes, followed by 10 cycles of 94° C. 15 seconds, 68° C. 30 seconds (decreasing by 0.3° C. per cycle) and 68° C. 10 minutes. Followed by 14 cycles of 94° C. 15 seconds, 65° C. 30 seconds and 68° C. 10 minutes.

Gel Electrophoresis/Southern Blotting

The products of the PCR reaction were resolved by agarose gel electrophoresis as follows. A 0.8% gel was prepared. Ideally the gel should be 20 cm or longer in order to allow sufficient resolution of long telomeres. Alternatively, depending upon the telomete length of the tissue under analysis, gel electrophoresis systems capable of resolving high molecular weight DNA fragments can be employed. These would include Field Inversion Gel Electrophoresis (FIGE) and Pulsed Field Gel Electrophoresis (PFGE). In this example, the DNA fragments were resolved by 0.8% agarose gel electrophoresis and FIGE. The FIGE, was carried out using a 1% agarose (SeaKem® Gold, FMC Bioproducts, Rockland, Me., USA) in 0.5×TBE, with the following switch conditions: 0.2-0.4 seconds (linear shape), forward voltage 180, reverse voltage 120 for 20 hours, with buffer recirculated at a temperature of 16 to 18° C. A ficol-based gel loading buffer was added to the PCR reactions and half of the 20 μl PCR reaction was loaded into the wells of the gel and a DNA size marker was also included. Electrophoresis took place over night at 70V (2.5 to 3 Volts per cm of gel length). The gel was ethidium bromide stained and the gel observed to check that products are sufficiently resolved. Standard laboratory southern blotting procedures were used to transfer the DNA from the gel onto a nylon membrane. In this case alkaline transfer was carried out by first depurinating the DNA by washing the gel for 10 minutes in 0.25 M HCl, and denaturation by washing the gel for 15 minutes in transfer buffer containing 0.5M NaOH and 1.5M NaCl. The DNA was transferred by capillary blotting onto a positively charged nylon membrane (in this case Hybond N+ manufactured by Amersham/Pharmacia) for a minimum of 4 hours. The membrane was then neutralised by a 20 second wash in solution of 100 mM Tris-HCl, (pH 7.5) and NaCL 500 mM.

Detection of the Amplified Telomeric Fragments.

The DNA fragments were detected by hybridisation with a DNA probe containing the sequence of the subtelomeric DNA of the telomere of interest; in this case, DNA probes containing the sequence of the DNA adjacent to the XpYp telomere. It was also possible to detect the fragments by hybridisation with the TTAGGG repeat probe. The probes were labelled with ³²P using a standard random hexa-priming reaction, as provided in the commercially-available Amersham/Pharmacia Rediprime plus kit. Hybridisation was carried out at 60° C. overnight in a 15 ml of a buffer containing 500 mM Na₃HPO₄ (pH 7.2), 7% SDS, 1 mM EDTA and 1% BSA Following hybridisation, the membrane was washed in 0.1×SSC, 0.1% SDS at 60° C. until the wash solution did not contain detectable radioactivity. The hybridised fragments were detected by standard autoradiography or phospho-imaging.

PCR Primer Sequences.

Two alternative versions of telorette have been used, but Telorette 2 is about 20 times more efficient than Telorette, so the results shown are for Telorette 2. These linkers vary only in the design of the 3′ most bases:

| | |
|---|---|
| Telorette 1: | 5′-TGCTCCGTGCATCTGGCATCCCCTAAC-3′ |
| Telorette 2: | 5′-TGCTCCGTGCATCTGGCATCTAACCCT-3′ |
| Teltail: | 5′-TGCTCCGTGCATCTGGCATC-3′ |
| XpYpE2: | 5′- TTGTCTCAGGGTCCTAGTG -3′ |
| 12qA: | 5′-GGGACAGCATATTCTGGTTACC-3′ |

Results

This analysis is on the XpYp telomere:

Lanes 1-6 Human thyroid cancer celline (6 different clones)

Lane 7-9 Human sperm sample from 3 unrelated men.

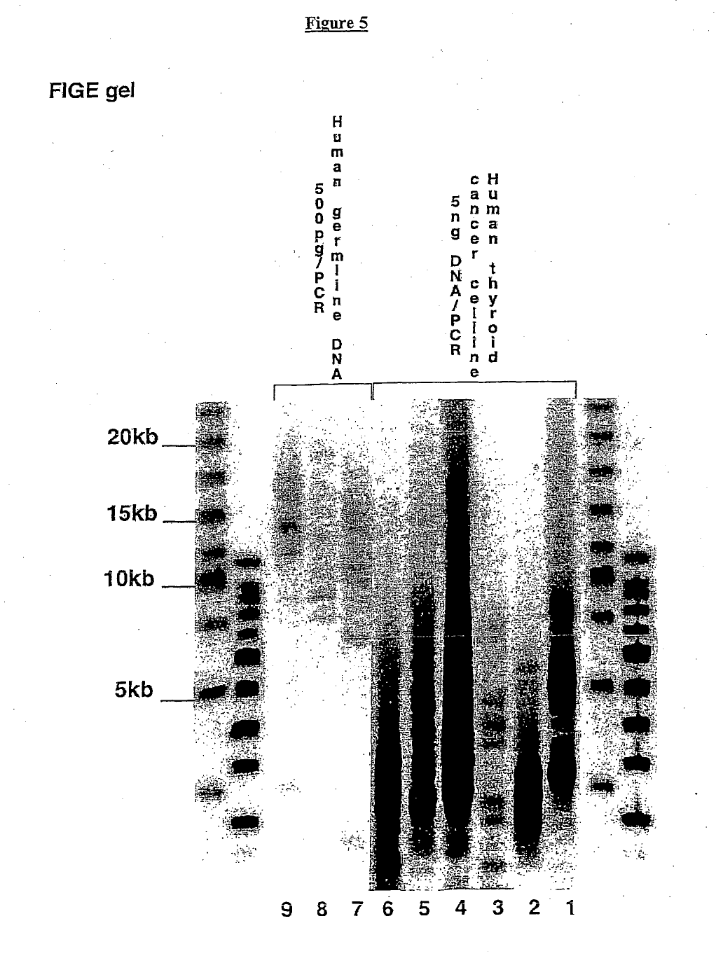

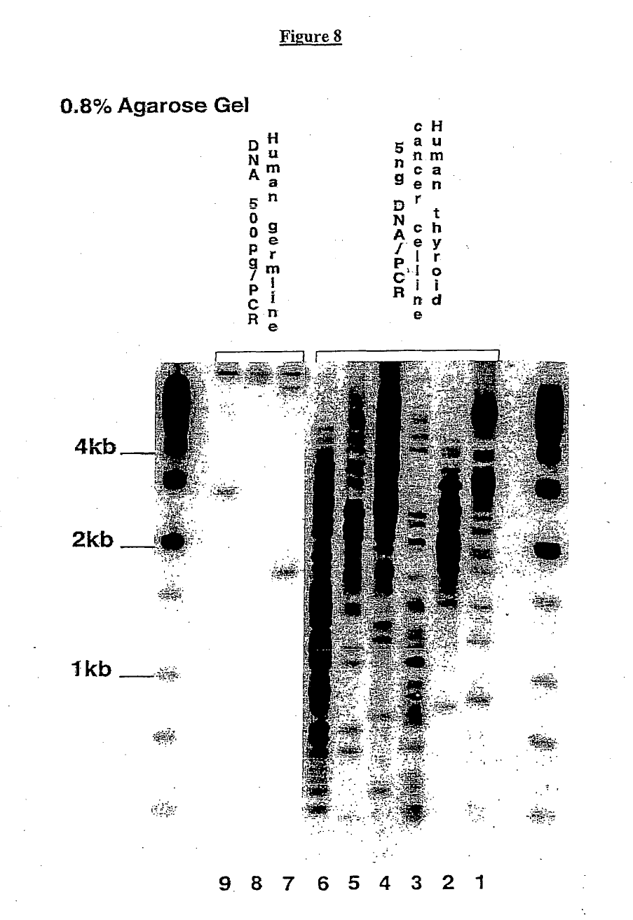

MTL analysis was carried out from the FIGE gel on lanes 1-2, 4-5, 7-9 (Lanes 3 and 6 were excluded because the lower molecular weight fragments were lost from the bottom of the FIGE gel, these fragments can be observed on the 0.8% Agarose gel). FIGE gel results are shown in FIG. 5 and 0.8% Agarose gel results are shown in FIG. 8. The results of the MTL analysis were as follows:

| | |
|---|---|
| Lane 1, | 3.30 kb |
| Lane 2, | 2.25 kb |
| Lane 3, | — |
| Lane 4, | 5.24 kb |
| Lane 5, | 2.07 kb |
| Lane 6, | — |
| Lane 7, | 11.23 kb |
| Lane 8 | 12.42 kb |
| Lane 9, | 14.58 kb |

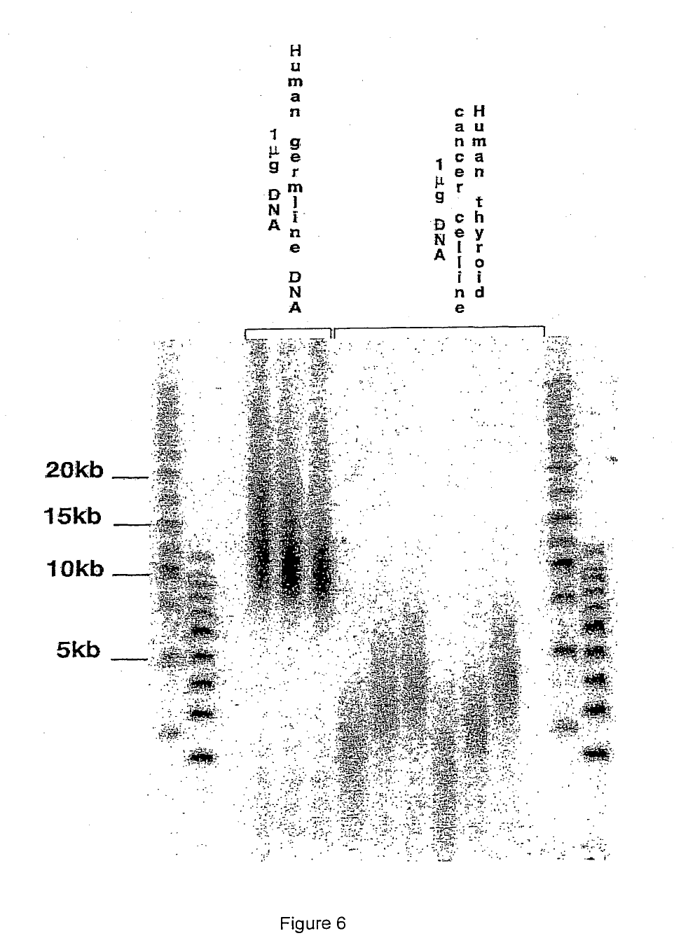

A comparison between the above results using the method of the invention (FIG. 5) and that using the conventional TRF analysis (FIG. 6) shows the higher resolution and clarity of the former.

### EXAMPLE 2

### Single Molecule Analysis

In addition, it is possible using the long-range PCR conditions described in Example 1 to determine telomere length from DNA obtained from human sperm. Human sperm DNA contains the longest telomeres observed in the human body, typically in the region of 10 to 18 kb in length. An example of PCR-based telomere length determination is shown in FIG. 7. Here, DNA obtained from human sperm was diluted as described in more detail below prior to the ligation step such that 4 ng, 1 ng and 250 pg of DNA were ligated to the “telorette” linker. The subsequent PCR reaction contained 1/10 of the ligation reaction, and therefore contains 400 pg, 100 pg and 25 pg of DNA. This represents 133, 33 and 8 haploid genome equivalents, respectively. In the 4 ng ligation reaction (400 pg PCR reaction) a smear of fragments is observed with an average length of 12 kb. As the amount of DNA in the ligation reaction is reduced, single fragments can be observed, these represent telomeres amplified from single molecules.

Serial Dilution

When using 500 ng of DNA in the ligation and therefore 50 ng of DNA in the PCR reaction, a smear correlating to telomere length as predicted from the standard telomere length analysis was obtained. However, this smear was overlaid by numerous other bands that hamper the interpretation of the results. The PCR conditions (ie number of cycles) coupled with detection of the products by hybridisation to a ³²P probe suggested that single molecules were being amplified. Therefore, the technique was too sensitive for the amount of DNA. Dilution experiments were carried out to reduce the amount of DNA in an attempt to reduce the additional banding pattern. The DNA was serially diluted to the point at which single amplification products were observed in some reactions (ie others contained no amplification products). The DNA was diluted serially ie 1 in 5 in Tris-HCl pH8.5, so that 100 ng of DNA was added to the 10 μl ligation reaction and the reaction then serially diluted 1 in 5 to provide a dilution series of 20 ng/μl, 4 ng/μl, 800 pg/μl, 160 pg/μl, 32 pg/μl, 6.4 pg/μl and 1.3 pg/μl. The haploid human genome weighs 3 pg, therefore the final dilution in this series would contain less than one molecule per μl.

PCR reactions were carried out, as described above, on each of these series of dilutions to monitor the dilution. The effect of the dilution is that, at higher input DNA amounts, the reaction results in a smear of hybridising fragments, but at lower dilutions this smear fragments into single hybridising bands. This effect is observed in FIG. 7 hereinbelow; in this case a 12 kb germline telomere smear observed in the 400 pg/μl dilutions, which fragments into 2 bands at the 25 pg/μl dilution. These experiments indicate that single molecule amplification can be achieved.

Therefore, the method of this invention is designed to detect telomere length at the single molecule level and is sufficient for determining the average telomere length in a small sample. Standard telomere length analysis of the human germline reveals that telomere length is 12 kb. However, single molecule analysis reveals many additional telomeres much shorter than 12 kb, as shown in FIG. 7. Here, the majority of the bands are around the 12 kb size and these form a smear in the 400 pg PCR reaction. However, additional, small bands are observed of around 2.2 kb, 4 kb and 7 kb. Telomeres of this length are not observed using conventional telomere length analysis.

Other Results

This experiment also demonstrates that telomeres can be detected in DNA that has been solubilised by restriction enzyme digestion (this facilitates a more accurate DNA concentration measurement). In addition, this experiment included a Mung bean nuclease treatment prior to ligation. This treatment renders the telomeric terminus blunted-ended, and effectively prevents the ligation reaction, thereby demonstrating the requirements for base-pairing of the telorette linker to the 3′ strand.

### EXAMPLE 3

### Modifications

(a) The method of Examples 1 and 2 was carried out as described, using the following oligonucleotides, which can be included in equimolar amounts in the ligation reaction to the same final concentration as detailed in the above Examples. These telorettes are identical, apart from the seven 3′ bases, which vary such that all the possible six positions within the telomere repeat sequence can be covered.

| | |
|---|---|
| Telorette 1: | 5′-TGCTCCGTGCATCTGGCATCCCCTAAC-3′; |
| and | |
| Telorette 2: | 5′-TGCTCCGTGCATCTGGCATCTAACCCT-3′ |

as in Example 1, plus the following additional telorettes:

| | |
|---|---|
| Telorette 3: | 5′-TGCTCCGTGCATCTGGCATCCCTAACC-3′ |
| Telorette 4: | 5′-TGCTCCGTGCATCTGGCATCCTAACCC-3′ |
| Telorette 5: | 5′-TGCTCCGTGCATCTGGCATCAACCCTA-3′ |
| Telorette 6: | 5′-TGCTCCGTGCATCTGGCATCACCCTAA-3′ |

It was found that Telorettes 2, 3 and 4 have similar efficiencies of about 10%, whereas the other telorettes have efficiencies of about 0.43%.

(b) Another modification is to use a DNA polymerase to fill in any gaps between the oligonucleotide and the 5′ end of the telomeric strand. This could employ any DNA polymerase lacking 5′ to 3′ exonuclease activity. The filling-in reaction may be carried out at the ligation step by the incorporation of the DNA polymerase (eg 1 unit of the Klenow fragment of DNA polymerase I, supplied by Amersham/Pharmacia) plus dCTP, dATP and dTTP (all at conc. of 0.02 mM, supplied by Promega) into the ligation reaction itself (provided that the ligation buffer is compatible with the DNA polymerase).

### EXAMPLE 4

### Single Telomere Length Analysis (STELA)

Material and Methods

Cell Culture

Fibroblast strains IMR-90, IMR-91, WI-38, AG08049, AG08048, AG11241, AG07119A, AG10937 and AG10938 were obtained from the Coriell Cell Repository (Camden, USA). MRC-5 human diploid fibroblasts were obtained from the ECACC (European Collection of Cell Cultures, Porton Down, UK). HCA2 fibroblasts, HCA2-hTERT (Wyllie, F. S. et al. Nat. Genet. 24, 16-17 (2000).), MRC5 hTERT (McSharry, B. P., et al J. Gen. Virol. 82, 855-63 (2001)) and K1 human thyroid cancer cell line (Jones, C. J. et al. Exp. Cell Res. 240, 333-339 (1998).).

All cells were cultured in Eagle's minimum essential medium supplemented with Earle's salts containing 2× non-essential amino acids; 15% (v/v) foetal calf serum; 1×10⁵ IU/I penicillin; 100 mg/l streptomycin; and 2 mM glutamine. The onset replicative senescence was determined by at least 2 weeks of no cell growth and confirmed by BrdU labelling indexes <1% as described by Bond, J. A. et al in Mol. Cell Biol. 19, 3103-3114 (1999).

DNA Extraction and PCR

Cells were trypsinised and washed in PBS, and genomic DNA was extracted by standard Proteinase K, RNaseA, Phenol/Chloroform protocols (Sambrook et al, T. Molecular Cloning: A Laboratory Manual. 2^{nd} Edition, Cold Spring Harbor Laboratory Press, CITY (1989)). The DNA was solublised by digestion with EcoRI, quantified by Hoechst 33258 fluorometry (BioRad, Hercules, USA) and diluted to 10 ng/μl in 10 mM Tris-HCl pH 7.5. The DNA was ligated at 35° C. for 12 hours, in a 10μl reaction containing 10 ng genomic DNA, 0.9 μM Telorette Linker according to the invention (see below), 0.5 units of T4 DNA ligase (Amersham Biosciences, Little Chalfont, UK) and 1× the manufactures ligation buffer.

As a control, the 5′ overhang was removed by digestion of 2 μg of genomic DNA with 40 Units of Mung Bean nuclease (Amersham Biosciences, Little Chalfont, UK) and 1× the manufacturer's nuclease buffer. Following Phenol/Chloroform extraction, the DNA was ethanol-precipitated and washed in 70% ethanol, then re-suspended in 10 ml Tris-HCl pH8.0 and quantified by Hoechst 33258 fluorometry (BioRad, Hercules, USA).

The ligated DNA was diluted to 250 pg/μl in H₂O. Multiple PCRs (typically between 9-18 reactions per sample) for each test DNA were carried out in 10 μl volumes containg in the range of from 100-250 pg of ligated DNA, 0.5 μM of the telomere adjacent and Teltail primers according to the invention (see below), 75 mM Tris-HCl pH8.8, 20 mM (NH₄)₂SO₄, 0.01% Tween-20, 1.5 mM MgCl₂, and 1 Unit of a 25:1 mixture of Taq (ABGene, Epsom, UK) and Pwo polymerase (Roche Molecular Biochemicals, Lewes, UK). The reactions were cycled with an MJ PTC-225 thermocycler (MJ research, Watertown, USA) under the following conditions: 25 cycles of 94° C. 15 seconds, 65° C. (XpYpE2) or 66.5° C. (XpYp-415GC/AT allele specific primers) for 30 seconds and 68° C. for 10 mins.

The DNA fragments were resolved by 0.5% TAE agarose gel electrophoresis, and detected by Southen hybridisation with a ³²P labelled (Amersham Biosciences, Little Chalfont, UK) telomere adjacent probe generated by PCR between primers XpYpE2 and XpYpB2 (FIG. 9) and 500 pg of a probe to detect the 1 kb Molecular Weight markers (Stratagene, La Jolla, USA) comprising 25 ng of the 1 kb Molecular Weight markers (Stratagene, La Jolla, USA) 32P labelled (Amersham Biosciences, Little Chalfont, UK) The hybridised fragments were detected by phospho-imaging with a Molecular Dynamics Storm 860 phospho-imager (Amersham Biosciences, Little Chalfont, UK). The molecular weights of the DNA fragments were calculated using the Phoretix 1D quantifier (Nonlinear Dynamics, Newcastle upon Tyne, UK).

The oligonucleotide sequences were as follows:

| | |
|---|---|
| XpYpE2: | 5′- TTGTCTCAGGGTCCTAGTG -3′ |
| XpYpB2: | 5′- TCTGAAAGTGGACC(AT)ATCAG -3′ |
| XpYp-415GC: | 5′- GGTTATCGACCAGGTGCTCC -3′ |
| XpYp-415AT: | 5′- GGTTATCAACCAGGTGCTCT -3′ |
| Telorette1: | 5′- TGCTCCGTGCATCTGGCATCCCCTAAC -3′ |
| Telorette2: | 5′- TGCTCCGTGCATCTGGCATCTAACCCT -3′ |
| Telorette3: | 5′- TGCTCCGTGCATCTGGCATCCCTAACC -3′ |
| Telorette4: | 5′- TGCTCCGTGCATCTGGCATCCTAACCC -3′ |
| Telorette5: | 5′- TGCTCCGTGCATCTGGCATCAACCCTA -3′ |
| Telorette6: | 5′- TGCTCCGTGCATCTGGCATCACCCTAA -3′ |
| Teltail: | 5′- TGCTCCGTGCATCTGGCATC -3′ |

Results

In summary, the results show the application of STELA, for the analysis of in vitro aged human diploid fibroblasts. Considerable allelic variation of up to 8 kb in TTAGGG repeat content was observed, which was abolished by the ectopic expression of telomerase (hTERT). Also noted was a gradual generation of telomere length heterogeneity, and that the shortest telomeric allele at senescence is individual-specific (1.2 kb to 7.4 kb) and includes telomeres that are virtually devoid of telomere repeats. STELA therefore represents a technology that will allow a full appraisal of the role of telomere repeat dynamics in numerous biological situations.

Data are given in Table 1, which shows summary of STELA data. For the primary fibroblast strains, only the data at the point of senescence is shown. a. STELA data generated from both alleles: the means of the upper and lower distributions was calculated by dividing the distributions on the basis of the overall mean, and calculating the mean of the separated distributions. The change in telomere length was calculated using the overall mean of the distribution. IMR-90 appeared to loose the majority of lower the distribution at senescence therefore it was not possible accurately to determine the mean of the lower distribution and the telomere erosion rate. b. Allele specific STELA data shown, demonstrating allele-specific changes in telomere length.

TABLE 1
Summary of STELA Data
a.

| | | | | | | Mean |
|---|---|---|---|---|---|---|
| | | MTL Upper | MTL Lower | Shortest | Allelic | D |
| Fibroblast strain | XpYp MTL | distribution | distribution | telomere | Difference | bp/PD |
| IMR-90 S PD54 | 4.931 | 5.413 | | 0.013 | | |
| | (±0.28) | (±0.21) | | | | |
| IMR-91 S PD39 | 7.357 | | | 0.033 | 0 | −88 |
| | (±0.67) | | | | | |
| WI-38 S PD49 | 5.497 | 7.992 | 2.598 | 0.163 | 5.394 | −20 |
| | (±0.73) | (±0.48) | (±0.44) | | | |
| HCA2-S PD64 | 4.633 | 7.672 | 1.513 | 0.059 | 6.159 | −33 |
| | (±0.44) | (±0.28) | (±0.22) | | | |
| MRC5-S PD55 | 3.432 | 6.095 | 1.289 | 0.045 | 4.806 | −59 |
| | (±0.42) | (±0.25) | (±0.14) | | | |
| MRC5 Cl.1 S | 3.256 | 6.491 | 1.004 | 0.081 | 5.487 | −83 |
| PD29 | (±0.56) | (±0.24) | (±0.09) | | | |
| MRC5 Cl.2 S | 2.828 | 5.977 | 1.144 | 0.049 | 4.833 | −116 |
| PD30 | (±0.25) | (±0.22) | (±0.07) | | | |
| MRC5 Cl.3 S | 2.023 | | | 0.061 | 0 | −88 |
| PD26 | (±0.13) | | | | | |
| MRC5 Cl.4 S | 4.803 | 6.815 | 3.390 | 0.082 | 3.425 | −34 |
| PD25 | (±0.49) | (±0.28) | (±0.38) | | | |
| MRC5 Cl.5 S | 4.378 | 8.229 | 1.897 | 0.617 | 6.332 | −32 |
| PD24 | (±0.63) | (±0.23) | (±0.12) | | | |
| MRC5 Cl.6 S | 4.005 | 6.540 | 2.660 | 1.370 | 3.879 | |
| PD15 | (±0.44) | (±0.28) | (±0.17) | | | |
| MRC5 Cl.7 S | 4.550 | 5.989 | 3.281 | 1.631 | 2.708 | |
| PD17 | (±0.34) | (±0.29) | (±0.19) | | | |

b.

| | GC | | AT | | | | | |
|---|---|---|---|---|---|---|---|---|
| | allele | | allele | | Shortest | Alleic | GC Allele | AT Allele |
| Fibroblast strain | mean | SD | mean | SD | telomere | Difference | Δ bp/PD | Δ bp/PD |
| MRC5-S PD55 | 5.735 | 1.56 | 1.179 | 0.52 | 0.061 | 4.556 | −32 | −102 |
| | (±0.39) | | (±0.10) | | | | | |
| MRC5 hTERT | 8.452 | 1.95 | 8.037 | 2.16 | 0.375 | 0 | | |
| PD200+ | (±0.55) | | (±0.66) | | | | | |
| MRC5 hTERT | 8.730 | 2.23 | 5.100 | 1.27 | 0.662 | 3.630 | | |
| Sub Cl.1 PD19 | (±0.68) | | (±0.29) | | | | | |
| MRC5 Cl.1 S | 6.249 | 1.31 | 0.995 | 0.31 | 0.366 | 5.254 | −99 | −72 |
| PD29 | (±0.43) | | (±0.09) | | | | | |
| MRC5 Cl.3 S | 1.900 | 0.51 | 2.246 | 0.68 | 0.024 | 0.346 | −49 | −122 |
| PD26 | (±0.14) | | (±0.19) | | | | | |
| MRC5 Cl.8 S | 4.288 | 0.37 | 1.688 | 0.45 | 0.508 | 2.600 | −104 | −92 |
| PD24 | (±0.14) | | (±0.14) | | | | | |
| AG08049 S PD 8 | 4.159 | 0.48 | 4.847 | 1.05 | 1.376 | 0.688 | | |
| | (±0.15) | | (±0.31) | | | | | |
| AG07119A S | 9.829 | 2.63 | 4.250 | 0.97 | 0.032 | 5.579 | | |
| PD21 | (±1.10) | | (±0.31) | | | | | |
| AG11241 S PD14 | 9.055 | 1.26 | | | | | | |
| | (±0.28) | | | | | | | |
| | 5.034 | 1.16 | | | 0.034 | 4.021 | | |
| | (±0.22) | | | | | | | |

This Example is further illustrated with reference to the following FIGS. 9 to 11, in which:

FIG. 9 comprises three parts: a. A diagrammatic representation of STELA at the XpYp telomere. b. STELA analysis of two K1 clones with controls, A&B=Clones 3&4 with Telorette 2 ligated, C&D=Clones 3&4 with an irrelevant linker ligated, E=Clones 3&4 ligated with no linker, F&G=Clones 3&4 5′ overhang removed by nuclease treatment and the DNA ligated with Telorette2. Four separate PCR reactions using the same ligation, the primers used are detailed above. The fragments were detected by southern hybridisation with the XpYp telomere adjacent probe. c. The sensitivity of STELA demonstrated by the dilution of the K1 input DNA, the amount of DNA in each reaction is detailed below A=clone 3 and B=clone 4.

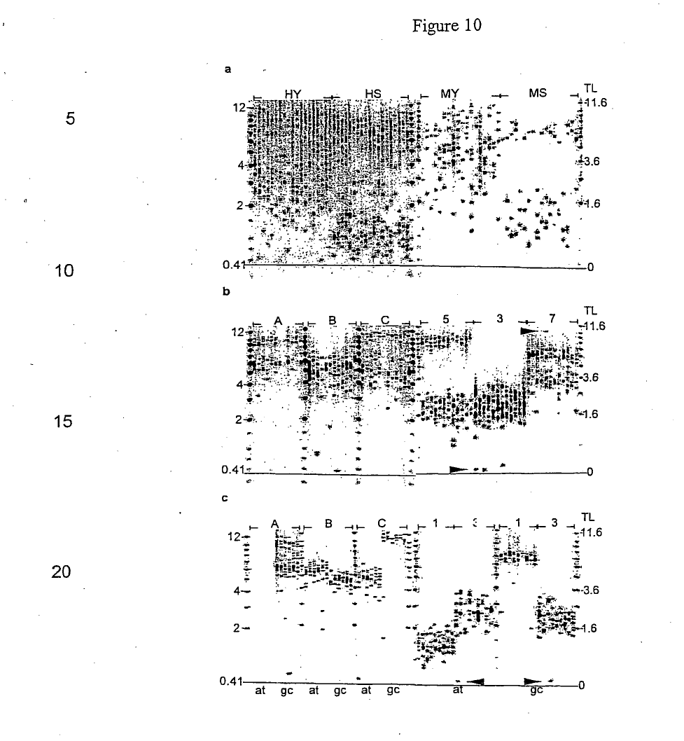

FIG. 10 comprises three parts: a STELA on young and senescent primary fibroblast strains, HY=HCA2 ‘Young’ PD28.5, HS=HCA2 Senescent PD64, MY=MRC5 ‘Young’ PD29, MS=MRC5 Senescent PD55. M=1 kb DNA Molecular Weight markers (Stratagene, La Jolla, USA), TL=Telomere length calculated from the 1^{st} telomere repeat. b. STELA on senescent primary fibroblasts derived from a pedgree, A=AG11241 (brother), B=AG08049 (Droband-male), C=AG0119A (mother). Senescent MRC5 clones 5, 3 and 7. c. Allele specific STELA. With the same DNAs used in b. A is homozygous for the GC Haplotype, B&C are heterozygous for the GC/AT haplotypes. Senescent MRC5 clones 1&3, MRC5 is heterozygous for the GC/AT haplotypes. Arrows in panels b and c indicate telomere length >3SD from their respective means.

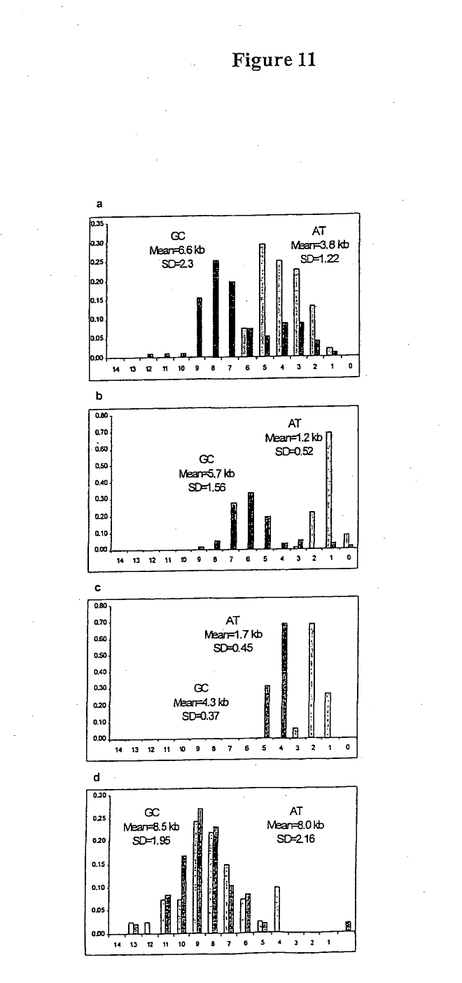

FIG. 11 comprises four histograms a-d generated from allele specific STELA analysis of MRC5 cells, distributions associated with the AT allele are shown in blue and the GC allele in red. X-axis show telomere size in kilobases, the Y-axis shows the relative proportions, telomere sizes were binned into 1 kb intervals. a. MRC5 ‘Young’ PD29. b. MRC5 Senescent PD55. c. MRC5 Clone 8 Senescent PD24. d. MRC5 hTERT PD200+.

1-30. (canceled).

31. A method for determining telomere length of mammalian chromosomal DNA, which method comprises the steps of:
(a) annealing a 3′ end of a single-stranded oligonucleotide linker or telorette to a single-stranded overhang of a telomere comprising a G-rich telomere strand having TTAGGG repeat sequences, and covalently binding the telorette to a 5′ end of a C-rich telomeric strand having CCCTAA repeat sequences thereby forming a ligation product;
(b) amplifying the ligation product to form at least one primer extension product; and
(c) detecting a length of the primer extension product.

32. The method according to claim 31, wherein the amplification step is carried out using:
(i) a first primer capable of annealing to a telomere-adjacent region of DNA but not capable of annealing to the C-rich telomeric repeat sequence CCCTAA; and
(ii) a second or teltail primer identical to the 5′ end sequence of the telorette;
wherein the amplification step is effected under conditions such that the first primer hybridises to the C-rich telomeric strand comprising CCCTAA repeats and is extended to form a first primer extension product, and further wherein the teltail primer hybridises to the first primer extension product and is extended to form a second primer extension product.

33. The method according to claim 32, wherein the teltail primer comprises a unique sequence of the telorette.

34. The method according to claim 31, wherein the telorette comprises a single-stranded oligonucleotide having from about 6 to about 12 nucleotides at the 3′ end which are complementary to and capable of annealing to a human telomeric repeated sequence TTAGGG.

35. The method according to claim 34, wherein the telorette further comprises a sequence of from 15 to 30 bases at the 5′ end, said sequence lacking substantial homology to human DNA sequences.

36. The method according to claim 34, wherein the 3′ bases of the telorette complementary to the telomere are short enough that, at an annealing temperature of at least about 35° C., the telorette is not capable of initiating strand synthesis.

37. The method according to claim 35, wherein the remaining 5′ bases of the telorette comprise a sequence lacking substantial homology to human DNA sequences, and which is efficient for PCR amplification only in the presence of first strand synthesis, initiated from either a primer capable of annealing to the sub-telomeric DNA or the variant repeats in the proximal regions of the telomere.

38. The method according to claim 31, wherein the telomere is a human telomere comprising the canonical telomere repeat sequence TTAGGG, and optionally telomere repeat variants selected from the group consisting of TGAGGG, TCAGGG, and TTGGGG, and any mixture thereof.

39. The method according to claim 38, wherein the telomere is that of human chromosomal DNA.

40. The method according to claim 31, wherein the telorette is ligated to the 5′ end of the C-rich telomeric strand using a ligase capable of joining juxtaposed DNA molecules between the 5′-phosphate and 3′-hydroxy groups.

41. The method according to claim 31, wherein the telorette is selected from at least one single-stranded oligonucleotide consisting of the group of sequences reading from 3′ to 5′ AATCCC, ATCCCA, TCCCAA, CCCAAT, CCAATC and CAATCC, or any mixture thereof.

42. The method according to claim 31, wherein the ligation reaction is carried out enzymatically at a temperature of from about 35° C. to about 37° C.

43. The method according to claim 42, wherein, following the ligation step, the reaction temperature is raised sufficiently to heat-inactivate the ligase enzyme.

44. The method according to claim 31, wherein the ligation product is amplified by PCR.

45. The method according to claim 44, wherein the amplification step is performed under long-range PCR conditions.

46. The method according to claim 44, wherein the amplification step is carried out at a temperature of from about 60° C. to about 70° C.

47. The method according to claim 31, wherein the DNA sample has a weight of from about 10 pg to about 1000 pg.

48. A kit for determining telomere length of mammalian chromosomal DNA, comprising at least one telorette sequence according to claim 31, at least one teltail primer identical to the 5′ end of the telorette, or any mixture thereof.

49. The kit according to claim 48, wherein the teltail primer comprises a unique sequence of the telorette.

50. The kit according to claim 48, further including at least one chromosome-specific primer, at least one allele-specific primer, at least one hybridisation probe, or any mixture thereof.

51. The kit according to claim 50, further including at least one ligase, at least one component specific for long-range PCR, at least one buffer, or any mixture thereof.

52. The kit according to claim 51, further including computer software and/or a spreadsheet adapted for calculation of mean telomere length.

53. The kit according to claim 52, further including instructions for using said kit to calculate a mean telomere length.

54. A primer for use in determining telomere length of mammalian chromosomal DNA, comprising a 3′ terminal having 6 to 12 nucleotide bases which are complementary to and capable of annealing to a G-rich telomeric overhang, and a 5′ terminal comprising 15 to 30 bases which are selected so as not to be complementary to the telomere but to be suitable for PCR amplification.

55. The primer according to claim 54, wherein said primer is selected from the group of sequences consisting of:

| | |
|---|---|
| XpYp-415GC: | 5′-GGTTATCGACCAGGTGCTCC-3′; |
| XpYp-415AT: | 5′-GGTTATCAACCAGGTGCTCT-3′; |
| XpYpE: | 5′-GCGGTACCTAGGGTTGTCTCAGGGTCC-3′; |
| 12qA: | 5′-GGGACAGCATATTCTGGTTACC-3′; |
| XpYpEorang: | 5′-CTGTCTCAGGGTCCTAGTG-3′; |
| XpYpE2: | 5′-TTGTCTCAGGGTCCTAGTG-3′; |
| XpYpB2: | 5′-TCTGAAAGTGGACC(AT)ATCAG-3′; |
| Telorette1: | 5′-TGCTCCGTGCATCTGGCATCCCCTAAC-3′; |
| Telorette2: | 5′-TGCTCCGTGCATCTGGCATCTAACCCT-3′; |
| Telorette3: | 5′-TGCTCCGTGCATCTGGCATCCCTAACC-3′; |
| Telorette4: | 5′-TGCTCCGTGCATCTGGCATCCTAACCC-3′; |
| Telorette5: | 5′-TGCTCCGTGCATCTGGCATCAACCCTA-3′; |
| Telorette6: | 5′-TGCTCCGTGCATCTGGCATCACCCTAA-3′; |
| Teltail: | 5′-TGCTCCGTGCATCTGGCATC-3′; |
| 12qA: | 5′-GGGACAGCATATTCTGGTTACC-3′; |
| 7qA: | 5′-GGGACAGCATATTCTGGTTTCC-3′; |
| and | |
| Nitu14eD: | 5′-CTCTGAGTCAGGAGCGTCTCC-3′. |

56. A method for determining telomere length of mammalian chromosomal DNA as described in claim 31, for use in assessing a potential anti-cancer treatment or other cancer related procedure, for use in the analysis of a biopsy sample, for assessing the effect of stem cells in bone marrow transplantation, for assessing telomere dynamics with age, for assessing the effect of modulating telomerase activity, or for assessing, treating, or diagnosing male infertility.

57. A kit for determining telomere length of mammalian chromosomal DNA as described in claim 48, for use in assessing a potential anti-cancer treatment or other cancer related procedure, for use in the analysis of a biopsy sample, for assessing the effect of stem cells in bone marrow transplantation, for assessing telomere dynamics with age, for assessing the effect of modulating telomerase activity, or for assessing, treating, or diagnosing male infertility.

58. A primer for determining telomere length of mammalian chromosomal DNA as described in claim 54, for use in assessing a potential anti-cancer treatment or other cancer related procedure, for use in the analysis of a biopsy sample, for assessing the effect of stem cells in bone marrow transplantation, for assessing telomere dynamics with age, for assessing the effect of modulating telomerase activity, or for assessing, treating, or diagnosing male infertility.
